(19) United States
(12) Patent Application Publication
(10) Pub. No.: US 2018/0089586 A1
(43) Pub. Date: Mar. 29, 2018

(54) ARTIFICIAL NEURAL NETWORKS FOR HUMAN ACTIVITY RECOGNITION

(71) Applicant: STMicroelectronics S.r.l., Agrate Brianza (IT)

(72) Inventors: Danilo Pietro PAU, Sesto San Giovanni (IT); Emanuele PLEBANI, Sotto il Monte Giovanni XXIII (IT)

(21) Appl. No.: 15/280,463

(22) Filed: Sep. 29, 2016

(51) Int. Cl. G06N 99/00; A61B 5/0205; A61B 5/11; A61B 5/00; G06N 3/04

(52) U.S. Cl. CPC G06N 99/005; A61B 5/0205; A61B 5/02055; A61B 5/02438; A61B 5/7264; G06N 3/04; A61B 5/1118

(57) ABSTRACT

Human activities are classified based on activity-related data and an activity-classification model trained using a classification-equalized training data set. A classification signal is generated based on the classifications. The classification-equalized training data set, may, for example, includes a first class having a first sequence length and a number of samples N, and one or more additional classes each having a respective sequence length tⱼ and a respective number of samples Nⱼ determined based on the number of samples N of the first class. For example, a respective sequence length tⱼ and a respective number of samples Nⱼ which satisfy: (i) Nⱼ>N, for sequence length tⱼ; and (ii) Nⱼ<N, for tⱼ−1. The activity-related data may include one or more of acceleration data, orientation data, position data, and physiological data.

### BACKGROUND

### Technical Field

The present disclosure relates to techniques, devices and systems for recognizing human activity based on sensed information, such as signals acquired by wearable sensor devices.

### Description of the Related Art

Wearable sensor device may continuously acquire and store information, and periodically transmit signals conveying the information, which may be processed in real time, for example, remotely by a host device or locally by the wearable device itself. Activities in which a wearer is engaged may be detected based on the information conveyed by the sensor signals. The signals may convey physiological data, acceleration data, rotation data, position data, etc.

The information may be analyzed to determine one or more activities in which the wearer is engaged. For example, a wearable sensor device may include one or more accelerometers, and an activity of the wearer may be determined based on accelerometer data from the one or more accelerometers, complemented by one or more gyroscopes. Simplest models may typically be employed to determine an activity of a wearer based on accelerometer data.

### BRIEF SUMMARY

In an embodiment, a method comprises: receiving activity-related data; determining activity classifications based on the received activity-related data and an activity-classification model trained using a classification-equalized training data set; and generating a classification signal based on the determined classifications. In an embodiment, for each class cⱼ other than a determined class cᵢ, a sequence length tⱼ such that the total number of samples Nⱼ for the class cⱼ, assuming the sequences of the class cⱼ are truncated at sequence length tⱼ, equals or just exceeds the number N of samples in the class cᵢ. In other words, Nⱼ<N for length tⱼ−1. Thus, in an embodiment, tⱼ for each class other than the determined class is determined based on the number of samples N in the determined class. In an embodiment, the classification-equalized training data set comprises a first class having a first sequence length and a number of samples N, and one or more additional classes each having a respective sequence length tⱼ and a respective number of samples Nⱼ which satisfy:

Nⱼ>N, for sequence length tⱼ; and

Nⱼ<N, for sequence length tⱼ−1.

In an embodiment, the method comprises: generating the classification-equalized training data set; and training the activity-classification model. In an embodiment, the determining activity classifications comprises extracting feature data based on the received activity-related data. In an embodiment, the determining comprises using an artificial neural network having a feed-forward architecture. In an embodiment, the artificial neural network comprises a layered architecture including one or more of: one or more convolutional layers; one or more pooling layers; one or more fully connected layers; and one or more softmax layers. In an embodiment, the artificial neural network comprises a finite state machine. In an embodiment, the determining comprises using an artificial neural network having a recurrent architecture. In an embodiment, the determining comprises using feature extraction and a random forest. In an embodiment, the determining comprises applying a temporal filter. In an embodiment, the activity-related data comprises one or more of: acceleration data, orientation data, geographical position data, and physiological data.

In an embodiment, a device comprises: an interface, which, in operation, receives one or more signals indicative of activity; and signal processing circuitry, which, in operation: determines activity classifications based on the received signals indicative of activity and an activity-classification model trained using a classification-equalized training data set; and generates a classification signal based on the determined classifications. In an embodiment, the classification-equalized training data set comprises a first class having a first sequence length and a number of samples N, and one or more additional classes each having a respective sequence length tⱼ and a respective number of samples Nⱼ which satisfy:

Nⱼ>N, for sequence length tⱼ; and

Nⱼ<N, for sequence length tⱼ−1.

In an embodiment, the signal processing circuitry comprises a feature extractor, a random forest and a temporal filter. In an embodiment, the signal processing circuitry comprises an artificial neural network having a feed-forward architecture. In an embodiment, the artificial neural network comprises a layered architecture including one or more of: one or more convolutional layers; one or more pooling layers; one or more fully connected layers; and one or more softmax layers. In an embodiment, the signal processing circuitry comprises an artificial neural network having a recurrent architecture. In an embodiment, the signal processing circuitry, in operation: generates the classification-equalized training data set; and trains the activity-classification model. In an embodiment, the signal processing circuitry comprises a finite state machine. In an embodiment, the one or more signals indicative of activity comprise signals indicative of one or more of: acceleration data, orientation data, geographical position data, and physiological data.

In an embodiment, a system comprises: one or more sensors, which, in operation, generate one or more activity-related signals; and signal processing circuitry, which, in operation: determines activity classifications based on activity-related signals and an activity-classification model trained using a classification-equalized training data set; and generates a classification signal based on the determined classifications. In an embodiment, the classification-equalized training data set comprises a first class having a first sequence length and a number of samples N, and one or more additional classes each having a respective sequence length tⱼ and a respective number of samples Nⱼ which satisfy:

Nⱼ>N, for sequence length tⱼ; and

Nⱼ<N, for sequence length tⱼ−1.

In an embodiment, the signal processing circuitry comprises a feature extractor, a random forest and a temporal filter. In an embodiment, the signal processing circuitry comprises an artificial neural network having a feed-forward architecture. In an embodiment, the signal processing circuitry comprises an artificial neural network having a recurrent architecture. In an embodiment, the one or more sensors include one or more of: an accelerometer; a gyroscope; a position sensor; and a physiological sensor.

In an embodiment, a system, comprises: means for providing activity-related data; and means for generating an activity classification signal based on activity-related data and an activity-classification model trained using a classification-equalized training data set. In an embodiment, the classification-equalized training data set comprises a first class having a first sequence length and a number of samples N, and one or more additional classes each having a respective sequence length tⱼ and a respective number of samples Nⱼ which satisfy:

Nⱼ>N, for sequence length tⱼ; and

Nⱼ<N, for sequence length tⱼ−1.

In an embodiment, the means for generating the activity classification signal comprises a memory and one or more processor cores, wherein the memory stores contents which in operation configure the one or more processor cores to generate the activity classification signal.

In an embodiment, a non-transitory computer-readable medium's contents configure signal processing circuitry to perform a method, the method comprising: receiving activity-related data; determining activity classifications based on the received activity-related data and an activity-classification model trained using a classification-equalized training data set; and generating a classification signal based on the determined classifications. In an embodiment, the classification-equalized training data set comprises a first class having a first sequence length and a number of samples N, and one or more additional classes each having a respective sequence length tⱼ and a respective number of samples Nⱼ which satisfy:

Nⱼ>N, for sequence length tⱼ; and

Nⱼ<N, for sequence length tⱼ−1.

In an embodiment, the method comprises: generating the classification-equalized training data set; and training the activity-classification model. In an embodiment, the determining activity classifications comprises extracting feature data based on the received activity-related data. In an embodiment, the determining comprises using an artificial neural network having a feed-forward architecture. In an embodiment, the artificial neural network comprises a layered architecture including one or more of: one or more convolutional layers; one or more pooling layers; one or more fully connected layers; one or more softmax layers. In an embodiment, the artificial neural network comprises a finite state machine. In an embodiment, the determining comprises using an artificial neural network having a recurrent architecture. In an embodiment, the artificial neural network comprises a finite state machine. In an embodiment, the determining comprises using feature extraction and a random forest. In an embodiment, the determining comprises applying a temporal filter.

### BRIEF DESCRIPTION OF THE SEVERAL VIEWS OF THE DRAWINGS

FIG. 1 is a functional block diagram of an embodiment of a system to detect user activities based on information gathered by a wearable device.

FIG. 2 illustrates an embodiment of a method of equalizing a number of samples in a data set.

FIG. 3 illustrates an embodiment of a classification pipeline to classify samples.

FIG. 4 illustrates an embodiment of a neuron structure.

FIG. 5 illustrates an embodiment of a structure of fully connected layer architecture.

FIG. 6 illustrates an embodiment of a logistic sigmoid function.

FIG. 7 illustrates an embodiment of a convolutional layer employing a sliding window.

FIG. 8 illustrates an embodiment of the structure of a convolutional network.

FIG. 9 illustrates an embodiment of a recurrent neural Echo State Network (ESN) pipeline.

FIG. 10 illustrates an embodiment of a convolutional neural network (CNN) pipeline.

FIG. 11 illustrates a portion of the embodiment of a CNN of FIG. 10 in more detail.

FIG. 12 illustrates an embodiment of a partial CNN computation process that may be employed by the embodiment of a CNN of FIG. 10.

FIG. 13 illustrates an embodiment of a logical diagram of a partial computation of a CNN network.

FIG. 14 illustrates an example distribution of a computational load of an embodiment.

FIG. 15 illustrates an example histogram of a label distribution on a window and a label selected by a median filter of an embodiment of a CNN pipeline.

FIG. 16 illustrates an embodiment of an encoding of a Finite State Machine (FSM), or a Finite State Automata (FSA).

FIG. 17 illustrates an embodiment of a convolutional neural network CNN pipeline which receives accelerometer and gyroscope data.

FIG. 18 illustrates an embodiment of a convolutional neural network CNN pipeline employing a global mean pooling layer.

### DETAILED DESCRIPTION

In the following description, certain details are set forth in order to provide a thorough understanding of various embodiments of devices, systems, methods and articles. However, one of skill in the art will understand that other embodiments may be practiced without these details. In other instances, well-known structures and methods associated with, for example, wearable devices and signal processing circuitry, such as transistors, multipliers, transmitters, NFC circuits, integrated circuits, etc., have not been shown or described in detail in some figures to avoid unnecessarily obscuring descriptions of the embodiments.

Unless the context requires otherwise, throughout the specification and claims which follow, the word “comprise” and variations thereof, such as “comprising,” and “comprises,” are to be construed in an open, inclusive sense, that is, as “including, but not limited to.”

Reference throughout this specification to “one embodiment,” or “an embodiment” means that a particular feature, structure or characteristic described in connection with the embodiment is included in at least one embodiment. Thus, the appearances of the phrases “in one embodiment,” or “in an embodiment” in various places throughout this specification are not necessarily referring to the same embodiment, or to all embodiments. Furthermore, the particular features, structures, or characteristics may be combined in any suitable manner in one or more embodiments to obtain further embodiments.

The headings are provided for convenience only, and do not interpret the scope or meaning of this disclosure.

The sizes and relative positions of elements in the drawings are not necessarily drawn to scale. For example, the shapes of various elements and angles are not drawn to scale, and some of these elements are enlarged and positioned to improve drawing legibility. Further, the particular shapes of the elements as drawn are not necessarily intended to convey any information regarding the actual shape of particular elements, and have been selected solely for ease of recognition in the drawings.

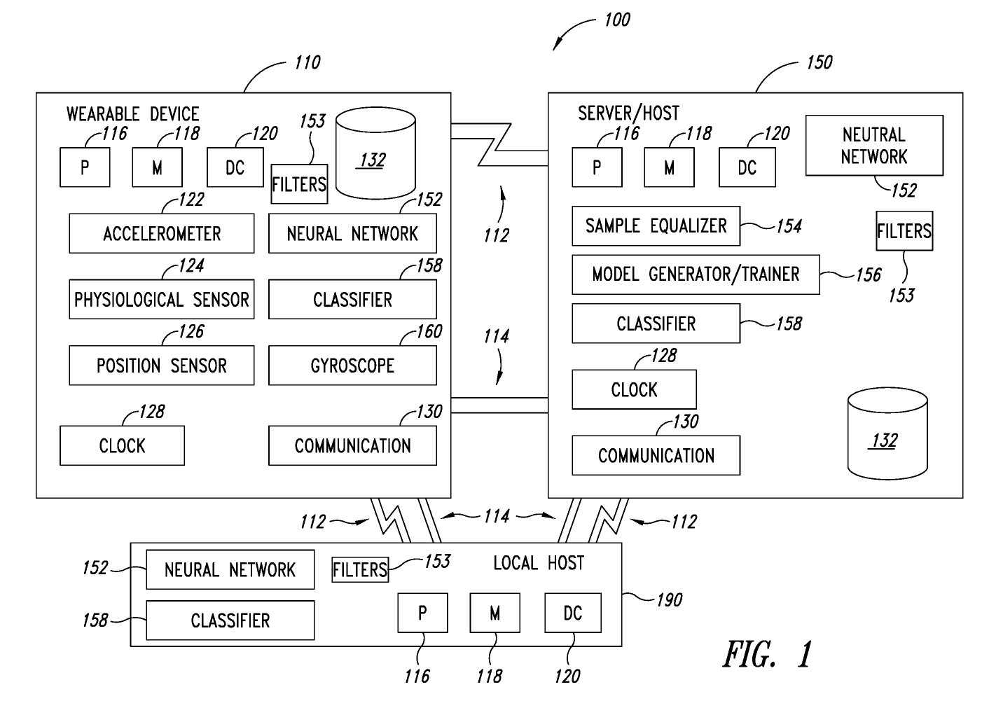

FIG. 1 is a functional block diagram of an embodiment of a system 100 to determine an activity of a wearer of a wearable device. As illustrated, the system 100 comprises a wearable sensor device 110, a host/server 150, and an optional local host 190. The wearable sensor device 110, the host/server 150, and the optional local host 190 as illustrated are communicatively coupled together by one or more wireless communication links 112 and one or more wired communication links 114, which may be used alone or in various combinations to facilitate transmission of control and data signals between the wearable device 110, the host/server 150 and the optional local host 190. The optional local host may facilitate communication between the wearable device 110 and the host/server 150, for example, when the host/server 150 is a remote server. As illustrated, the wearable sensor device 110, the host/server 150 and the optional local host include circuitry comprising one or more processors or processor cores P 116, one or more memories M 118, and discrete circuitry DC 120, such as one or more adders, one or more multipliers, one or more filters (such as band pass, low pass, high pass filters, infinite impulse response (IIR), finite impulse (FIR) filters, etc.), etc., which may, alone or in various combinations, implement one or more functions of the system 100.

As illustrated, the wearable device 110 comprises circuitry including one or more accelerometers 122, such as a triaxle accelerometer, one or more gyroscopes, such as the illustrated triaxle gyroscope 160, etc., one or more physiological sensors 124, such as a heart rate sensor, a temperature sensor, a respiration sensor, etc., one or more position sensors 126, such as a global position sensor, etc., one or more clocks 128, one or more communication circuits 130, and one or more database structures 132. For ease of illustration, other circuitry, which may typically be included in a wearable device such as the wearable device 110, are omitted from FIG. 1. Such circuitry may include bus systems, power systems, interfaces, etc.

The one or more accelerometers 122 sense movement data and generate signals indicative of the movement data, such as acceleration data for three axis of movement x, y and z of the wearable device 110. Multiple accelerometers 122 may be employed, such as an accelerometer for each axis, an accelerometer configured to gather acceleration data along two axes, together with an accelerometer to gather acceleration data along a third axis, an accelerometer configured to gather acceleration data along three axes, etc., and various combinations thereof.

The one or more physiological sensors 124 gather physiological data, such as heart rate information, temperature information, respiration information, etc., related to a wearer of the wearable device 110. The one or more position sensors 126 gather position data, such as a data related to a geographical position of the wearable device 110, data related to a position of the wearable device 110 on a wearer, etc. The one or more clocks 128 provide timing signals to facilitate the collection of data samples, for use, for example, in generating training data, determining wearer activities associated with the data samples, time stamps etc. The one or more communication circuits 130 facilitate the exchange of data and control signals between components of the system 100, and may include, for example, near-field communication circuits, serial bus communication circuits, LAN and WAN communication circuits, wi-fi communication circuits, mobile telephone communication circuits, etc., and various combinations thereof. The one or more data structures 132 may store, for example, training data, test data, control parameters, such as modeling parameters, instructions (for example, for execution by the one or more processors P 116), etc., and various combinations thereof.

The one or more gyroscopes 160 sense orientation data and generate signals indicative of the orientation data, such as orientation data with respect to three axis of a three-dimensional space x, y and z of the wearable device 110. Multiple gyroscopes 160 may be employed, such as a gyroscope for each axis.

As illustrated, the server/host 150 comprises circuitry including a neural network 152, one or more filter banks 153, such as IIR filters, FIR filters, etc., a sample equalizer 154, a model generator/trainer 156, one or more data structures 132, a classifier 158, which may classify a data sample based on a model generated by the model generator 156 and/or data stored in the one or more data structures 132 as being associated with one or more activities (e.g., stationary, walking, fast walking, jogging, biking, driving, etc.), one or more clocks 128 and communication circuitry 130. For ease of illustration, other circuitry, which may typically be included in a server/host such as the server/host 150, are omitted from FIG. 1. Such circuitry may include bus systems, power systems, interfaces, etc.

In some embodiments, one or more functions of the wearable device 110, of the server/host 150 and/or of the optional local host 190 may instead or also be performed by another of the wearable device 110, the server/host 150 and/or the optional local host 190. For example, in some embodiments, the wearable device and/or the local host may include a neural network, such as the neural network 152, and a classifier, such as the classifier 158, similar to those of the server/host 150.

The inventors have recognized that the number of samples in training data, such as data used to train a neural network to classify data samples, as well as the number of samples in test data sets to be classified (which may also be employed to update models) may tend to be skewed in favor of activities of a particular class. Table 1, below, illustrates the number of samples associated with various activity classes in an example training data set, referred to herein after as Dataset 1.1, Training Set.

TABLE 1
Example Dataset 1.1, Training Set Distribution

| Activity Class | Number of Samples | Percentage of Total Samples |
|---|---|---|
| Stationary | 4,536,217 | 46.44 |
| Walking | 1,448,165 | 14.83 |
| Jogging | 1,679,551 | 17.20 |
| Biking | 594,737 | 6.09 |
| Driving | 1,508,601 | 15.45 |

Table 2, below, illustrates a number of samples associated with various activity classes for a first test data set, referred to hereinafter as Dataset 1.1, Test Set 1.

TABLE 2
Example Dataset 1.1, Test Set 1 Distribution

| Activity Class | Number of Samples | Percentage of Total Samples |
|---|---|---|
| Stationary | 216,048 | 40.68 |
| Walking | 81,018 | 15.25 |
| Jogging | 81,018 | 15.25 |
| Biking | 72,016 | 13.56 |
| Driving | 81,018 | 15.25 |

Table 3, below, illustrates a number of samples associated with various activity classes for a second test data set, referred to hereinafter as Dataset 1.1, Test Set 2.

TABLE 3
Example Data Set 1.1, Test Set 2 Distribution

| Activity Class | Number of Samples | Percentage of Total Samples |
|---|---|---|
| Stationary | 207,046 | 39.66 |
| Walking | 81,018 | 15.52 |
| Jogging | 81,018 | 15.52 |
| Biking | 72,016 | 13.79 |
| Driving | 81,018 | 15.52 |

The data sequences used to generate the training and test data sets were acquired at a frequency of 50 Hz, but other acquisition frequencies may be employed, such as, for example, 16 Hz. For training and test purposes, the data sequences were decimated at a 1:3 ratio, generating sequences having an acquisition frequency of approximately 16 Hz. The training data set in the example of Table 1 was generated from 434 data sequences organized into five classes: stationary, walking, jogging, biking and driving. The test data set in the example of Table 2 was generated from 59 acquired data sequences not included in the training data set, and organized into the same five classes. The test data set in the example of Table 3 was generated from 58 acquired data sequences, which may include sequences included in the training data set, and organized into the same five classes.

Table 4, below, illustrates a number of samples associated with various activities for cleaned-up test data set, and includes a column showing a number of samples in the training data set. The cleaned-up test data set in the example of Table 4 was generated from 1054 acquired data sequences and organized into the same five classes. The distribution of samples is skewed toward the stationary and walking sequences. For ease of reference, the cleaned up test data set of Table 4 will be referred to hereinafter as Dataset 2.0, Cleaned-Up Test Set 1.

TABLE 4
Dataset 2.0, Cleaned-Up Test Set 1 Distribution

| | Number | Percentage of | Training Data |
|---|---|---|---|
| Activity | of Samples | Total Samples | Set Samples |
| Stationary | 8,674,636 | 80.11 | 4,536,217 |
| Walking | 1,844,416 | 17.03 | 1,448,165 |
| Jogging | 23,350 | 00.22 | 1,679,551 |
| Biking | 49,052 | 00.45 | 594,737 |
| Driving | 237,372 | 02.19 | 1,508,601 |

In the training of a model, the training error is often computed as the sum of the errors of each sample. If, as in the case of both the first test data set, Dataset 1.1, Test Set 1, and the Dataset 2.0, Cleaned-Up Test Set 1, some classes are more represented, the training error promotes those classes to the detriment of the least represented classes. For example, in the case of the Cleaned-Up Test Set of Table 4, a True Positive Rate (TPR) of 80.11% can be reached by predicting “stationary” all the time. To facilitate avoiding this issue, the data set may be equalized, that is, the same number of samples (or approximately the same number of samples) may be selected for each class.

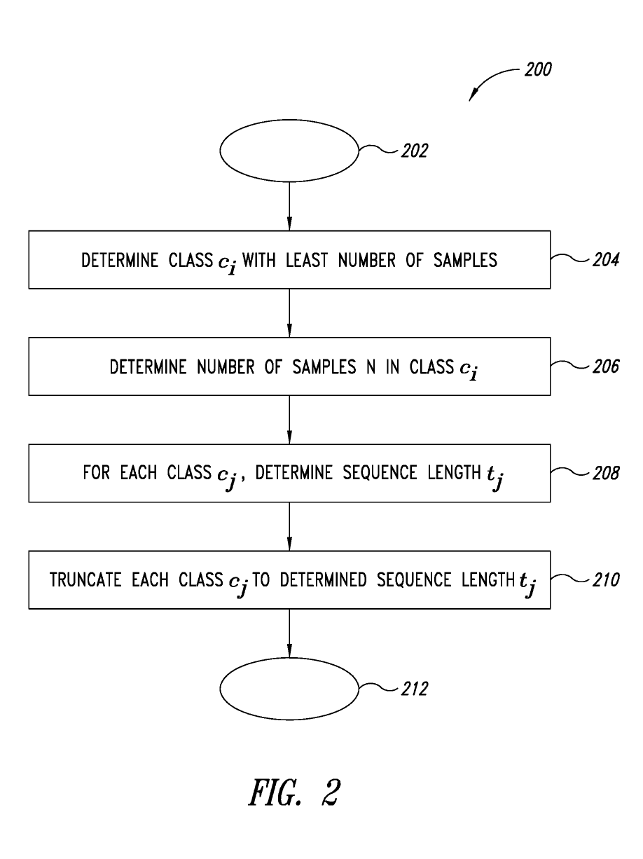

FIG. 2 illustrates an embodiment of a method 200, which may be employed by the system 100 of FIG. 1 (e.g., by the sample equalizer 154), to equalize the number of samples in a data set, such as a data set used for training or for evaluating the performing of the system 100. For convenience, FIG. 2 will be described with reference to the system 100 of FIG. 1 and the training data set of Table 1. Other embodiments of systems and devices may employ the method 200 of FIG. 2 with other data sets.

The method 200 proceeds from 202 to 204. At 204, the system 100 determines a class cᵢ having a fewest number of samples N. In the example of the training data set of Table 1, Biking would be selected as the class cᵢ having the fewest number of samples N.

The method 200 proceeds from 204 to 206. At 206, the system 100 determines the number of samples Nin the determined class cᵢ. In the example of the training data set of Table 1, the number of samples N in the Biking class cᵢ, would be determined to be 594,737.

The method 200 proceeds from 206 to 208. At 208, the system 100 determines, for each class cⱼ other than the determined class cᵢ, a sequence length tⱼ such that the total number of samples Nⱼ for the class cⱼ, assuming the sequences of the class cⱼ are truncated at sequence length tⱼ, equals or just exceeds the number N of samples in the class cᵢ. In other words, Nⱼ<N for length tⱼ−1. Thus, tⱼ for each class other than the determined class is determined based on the number of samples N in the determined class.

The method 200 proceeds from 208 to 210. At 210, the system 100, for each class cⱼ other than the determined class cᵢ, truncates all of the sequences in the class cⱼ to the determined sequence length tⱼ for the class cⱼ. In other words, samples in each sequence for each class cⱼ beyond the determined length tⱼ are discarded.

The method 200 proceeds from 210 to 212, where the system 100 may perform other processing, such as returning an equalized data set for use in training or evaluating a model, such as a neural network model.

Embodiments of the method 200 may include other acts not shown in FIG. 2, may not perform all of the acts shown in FIG. 2, and may perform the acts shown in FIG. 2 in various orders. For example, in some embodiments acts 204 and 206 may be combined. Equalizing the number of samples by truncating the sequence lengths facilitates discarding whole sequences during the sample selection process. Other methods of equalizing the number of samples may be employed in some embodiments. For example, other conditions may be applied to determine the sequence lengths to be applied. For example, at 208, the system 100 may instead determine, for each class cⱼ other than the determined class cᵢ, a sequence length tⱼ such that the total number of samples Nⱼ for the class cⱼ, assuming the sequences of the class cⱼ are truncated at sequence length tⱼ, is within a threshold number of the number N.

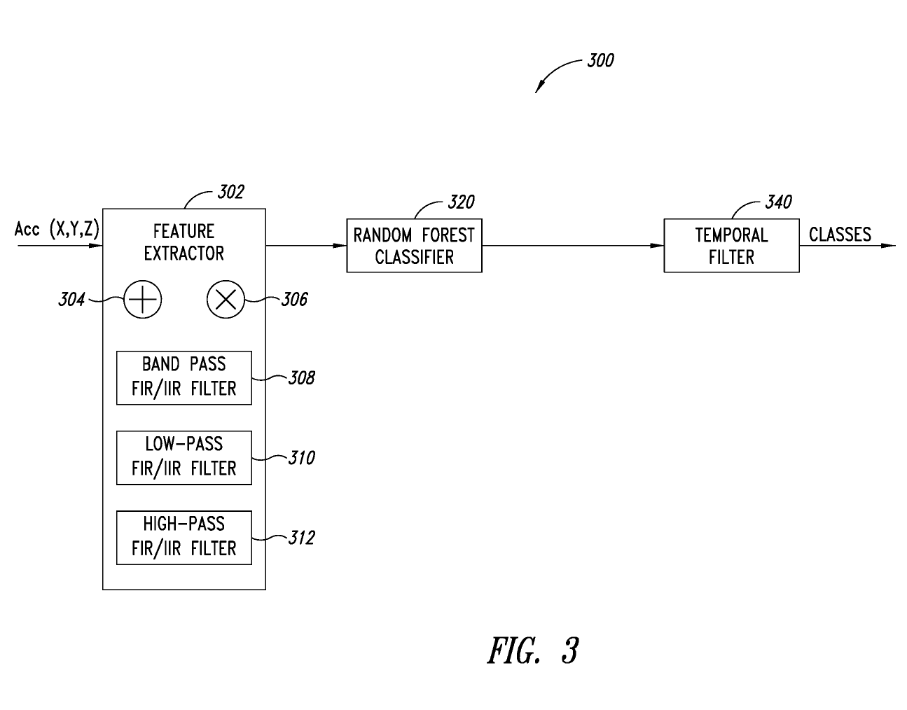

FIG. 3 illustrates an embodiment of a classification pipeline circuit 300 which may be employed, for example, by an embodiment of a system, such as the system 100 of FIG. 1 (e.g., by the classifier 158), to classify samples. For convenience, the classification pipeline 300 will be described with reference to the system 100 of FIG. 1 and with reference to acceleration data. Other embodiments of systems and devices may employ the classification pipeline 300 of FIG. 3 with other and/or additional activity-related data, such as acceleration data, orientation data, geographical position data, physiological data, etc., and various combinations thereof.

Acceleration data is received by a feature extractor 302. As illustrated, the feature extractor 302 includes one or more adders 304, one or more multipliers 306, one or more band-pass FIR/IIR filters 308, one or more low-pass FIR/IIR filters 310 and one or more high-pass FIR/IIR filters 312. Other mathematic operation circuits may be employed, such as dividers, square root operators, etc.

The acceleration data may be received by the feature extractor 302 in the form of input vectors a normalized with respect to a gravity acceleration g, with vectors a having a component in each of three axis and expressed in m/s² (e.g., a=[aₓ, a_{y}, a_{z}]). For convenience, vectors may be indicated with bold typeface herein. In an embodiment, the feature extractor computes an acceleration norm A for each received input as follows:

A=a·a

In an embodiment, the signal A is filtered with a band-pass FIR/IIR filter 308 and/or a low-pass FIR/IIR filter 310 and/or a high-pass FIR/IIR filter 312. As illustrated, the feature extractor provides extracted feature information to a random forest classifier 320 and to a temporal filter 340.

The random forest classifier 320 in an embodiment may comprise a multitude of decision trees, which discriminate classes. In an embodiment, the random forest trees may be implemented using complex sequences hard-coded if-then-else structures in C language. In an embodiment, the temporal filter 340 handles special cases based on the output of the random forest classifier 320 and corrects the output of the random forest classifier 320 in case of misclassification errors by filtering the current, future and previous classification samples (in time order) to output the correct class recognized: for example, some data at the output of 320 may indicate a classification output should be stationary, even if previous classification data indicates a different class. The output of the temporal filter may provide a more correct and coherent series of classifications of a current activity by exploiting temporal correlation of various previous decisions at the output of the random forest 320 that otherwise would be incoherent if left as they are. For example the random forest classifier 320 may output 6 (subsequent in time) classifications in the following sequence: running, running, driving, running, running, running etc. Clearly the 3rd classification output (driving) is highly incoherent with the previous and future ones and therefore will be replaced at the output of the temporal filter 340 with running so that the mistake is corrected.

Training of artificial neural network architectures on raw acceleration data was performed in a study in an end-to-end fashion without input sample pre-processing and feature design. Two architectures were evaluated: an Echo State Network (ESN) having a reservoir, three inputs (one for each acceleration axis) and an output trained through logistic regression; and a Convolutional Neural Network having four layers.

A typical neuron structure 400 of an embodiment is represented in FIG. 4. A set of inputs may comprise outputs of other neurons hᵢ, and input signals u, and are weighted by a set of values w₁, w₂, . . . wₖ₊₁, and then summed. The outputs of other neurons provides as input (the hᵢ values) may be, for example, generated exclusively by neurons in a previous level or layer in an Acyclic Connected Graph (ACG) structure of a feed forward network, generated from values computed in the same layer in a previous time step in a recurrent network, etc. The summed value is then subjected to a non-linearity function f(x) 402, as illustrated a sigmoid function as a non-limiting example since other functions may be used such as ReLU (Rectified Linear Unit), generating an output of the neuron y.

The outputs yⱼ of multiple neurons may be generated by stacking weights in a weight matrix W, determining a matrix product of the inputs and the weight matrix, generating as vector, and applying the nonlinearity element-wise on the vector. In an embodiment, a bias term b may be added to the neuron to compensate for the mean value of the input.

For a feed-forward network, the output of a neuron layer may be represented as:

yₗ=f(W·xₗ₋₁+b)

where xₗ₋₁ is the output of the previous layer (the input is x₀) and yₗ is the output of the current layer.

For a recurrent network, the output yₜ of a neuron layer may be represented as:

yₜ=f(Wₓ·xₜ+Wₕ·yₜ₋₁+b)

where xₜ is the input at the current time step, yₜ₋₁ is the output of the layer in the previous time step. The neuron input is split between the forward term Wₓ and the recurrent term Wₕ, which depends on the layer output in the previous time step.

A number of different nonlinearities f(x) may be employed in various embodiments. For example, a Rectified Linear Unit (ReLU) function may be employed. An example ReLU function returns zero for negative values, and does not change the value otherwise, and may be represented mathematically as follows:

f(x)=max(0,x).

A sigmoid function, such as a logistic function (S-shaped curve) with a mid-point at zero and a maximum of 1, may be employed. An example sigmoid function may be represented mathematically as follows:

$$f ( x ) = \frac{1}{1 + {e}^{- x}} .$$

A hyperbolic tangent function may be employed. An example hyperbolic tangent function may be represented mathematically as follows:

f(x)=tanh(x).

The ReLU nonlinearity may typically be employed in feed-forward networks, and the sigmoid or tanh nonlinearities may typically be employed in recurrent networks.

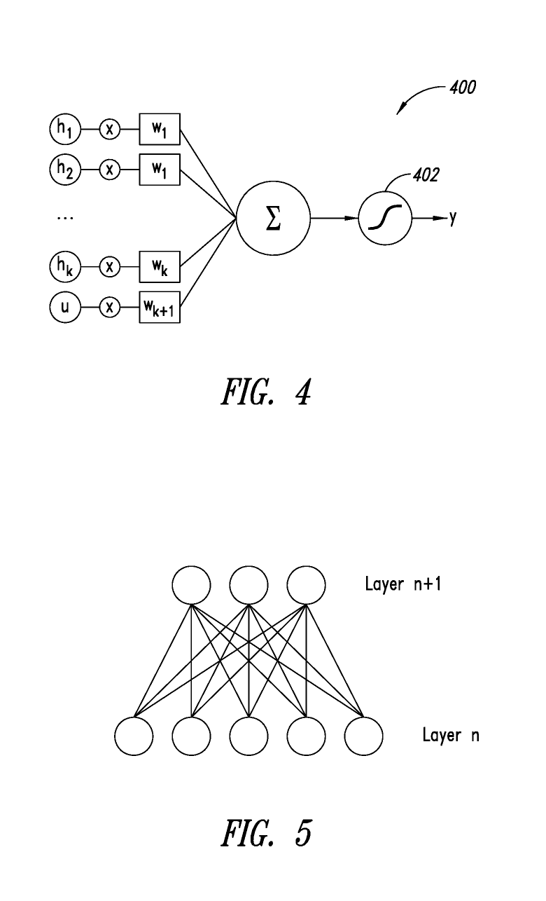

A Multi-Layer Perceptron (MLP) also known as fully connected architecture may be the simplest neural architecture, where a collection of neurons (layer) compute the output values from a previous (input) layer; the first layer is the input data, and the last layer is the prediction. Multiple layers can be stacked to compute increasing complex functions of the input. Because the output of a layer depends only on the values computed by the previous layer, the architecture is a feed-forward architecture. If a MLP is used as part of a larger network, each layer of the MLP may be referred to as a dense layer or a fully connected layer. FIG. 5 illustrates an example structure of a MLP architecture of an embodiment.

The sigmoid nonlinearity takes only values between 0 and 1 and thus can be interpreted as a probability estimate in a binary classification problem, e.g. a problem where only two classes are available. However, for multiple classes a tool which can estimate the probabilities over n classes is needed. In an embodiment, a softmax layer may be employed. In an embodiment, a softmax layer may be defined as:

$${y}_{j} = \frac{{e}^{x \cdot {w}_{j}}}{{\sum }_{i = 0}^{K} {e}^{x \cdot w} i} ,$$

where wⱼ·x is the output of a neuron just before the non-linearity. A softmax may thus be treated as 1) exponentiate the output of each neuron and 2) normalize the result across the whole layer to obtain probabilities. For two classes, the softmax reduces to the sigmoid function:

$${y}_{j} = \frac{{e}^{{w}_{{1}^{\cdot x}}}}{{e}^{{w}_{1} \cdot x} + {e}^{{w}_{2 \cdot x}}} = \frac{1}{1 + {e}^{( {w}_{2} - {w}_{1} ) \cdot x}} .$$

Thus the softmax may be treated as a generalization of the sigmoid/logistic function to multiple dimensions. FIG. 6 illustrates an example logistic sigmoid function.

The number of weights required to process an image or a window of a sequential signal grow as n² where n is the number of input values and thus may lead to overfitting. A convolutional layer may be employed to reduce the number of parameters, and facilitate avoiding overfitting. A convolutional layer may reduce the number of parameters through weight sharing and local connectivity. For example, in weight sharing neurons in layer n+1 may be connected to the neurons in layer n using the same set of weights. When local connectivity is employed, only the neurons in layer n near the location of the neuron in later n+1 are connected.

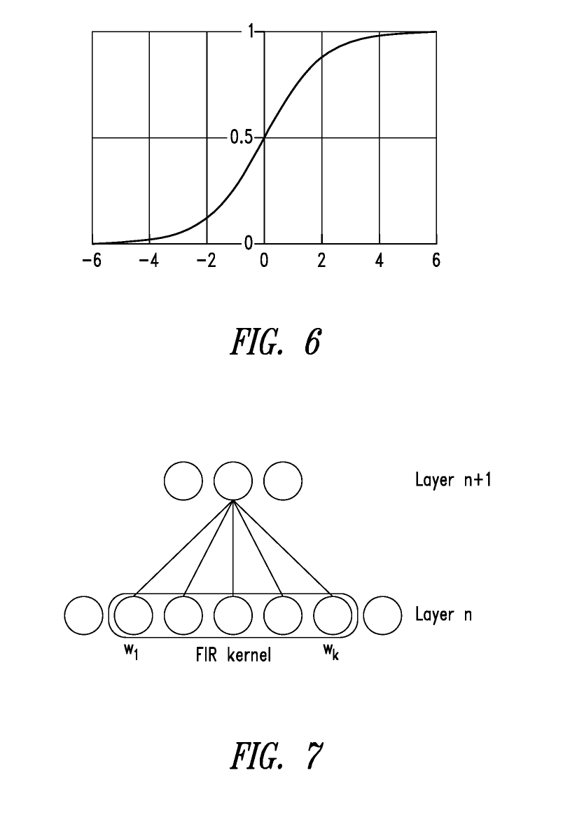

FIG. 7 illustrates an example of a convolutional layer employing a sliding window. Computing the weighted sum of the previous layer over a sliding window is equivalent to a FIR filtering operation on the previous layer; the layer is thus called convolutional. In the most general case, the layers n and n+1 have a number of channels, that is, values referred to the same spatial position (such as stereo channels in audio signals or color channels in images). The convolution is thus extended to all the input channels by applying a different filter to each channel and summing the results. Moreover, a number of output channels are generated by applying a different set of filters to the input channels. FIG. 8 shows the structure of an example convolutional network in a typical case. The filter weights are all learned, so in the design phase only three parameters are specified: the number of input channels c, the number of output channels m and the size of the filter kernel k.

A pooling layer may be employed to down-sample the input in order to introduce shift-invariance (insensitivity to small changes in alignment in the input signal) and to reduce the number of neurons in the subsequent layers. A pooling layer is often used after a convolutional layer and before a fully connected layer. The pooling layer is characterized by the window size N and a pooling function. Typical choices for the pooling function are the max function:

$$y = {max}_{i = 0 , … N - 1} {x}_{i}$$

and the average function:

$$y = \frac{1}{N} {\sum }_{i = 0}^{N - 1} {x}_{i}$$

where x is the output of the previous layer restricted to the pooling window.

An Echo State Network (ESN) may be employed in an embodiment as an alternative to the convolutional neural network. The ESN reservoir is characterized by a hidden state hₜ of size nᵣ and a weight matrix Wᵣ of size nᵣ×nᵣ. The network receives an input aₜ of size nᵢ weighted by a matrix Wᵢₙ of size nᵣ×nᵢ. Optionally, a bias bᵣ can be added to each neuron. The matrix Wᵣ may be computed by sampling the coefficients randomly from a uniform distribution in −1, 1: wᵢⱼ˜U([−1,1]) and randomly setting weights to zero with probability p, where p the sparsity of the weight matrix. For classification tasks, instead of directly classifying the hidden state, an average may be employed. To save memory, the exponential average y̅ₜ=(1−α)yₜ+αyₜ₋₁ may be used instead, where α is an averaging factor. The effective window size for averaging may be 1/α. The hidden state may be determined according to:

yₜ=[aₜhₜ]

where

hₜ=σ(Wᵢₙaₜ₋₁+Wᵣhₜ₋₁+bᵣ),

where σ is a nonlinearity (default may be the sigmoid function). The output class may be chosen as the maximum value after multiplying the hidden state by the output matrix Wₒ and adding the bias bₒ:

$$c = {max}_{i} ( {W}_{o} {\overline{y}}_{t} + {b}_{o} ) .$$

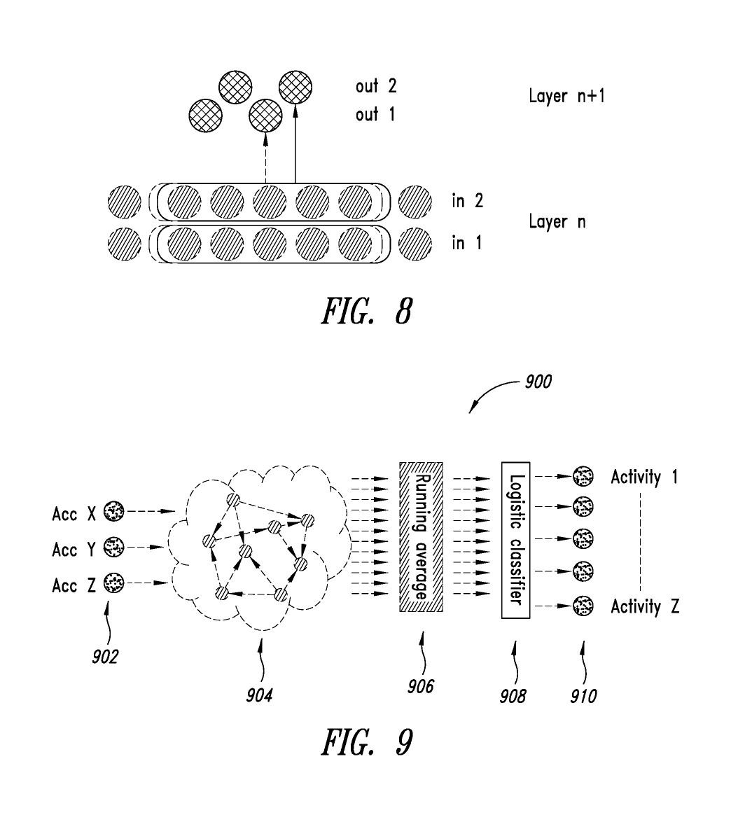

FIG. 9 illustrates an example embodiment of an ESN pipeline 900. For convenience, FIGS. 9 to 16 are described with reference to acceleration data. Various types of activity-related data may be employed in embodiments, such as acceleration data, orientation data, geographical position data, physiological data, etc., and various combinations thereof. Accelerometer data is received at 902 and provided to the reservoir network at 904. A running average is determined at 906, and logistic classification occurs at 908. The prediction is output at 910.

In neural network architectures the computations are dominated by scalar products, which combine ADD and MUL operations in a predictable pattern. The number of operations is discussed in terms of MADD for simplicity. The number of MUL is equal to the number of MADD, while the number of ADD is slightly lower. For each sample the hidden state is computed, requiring nᵣ·(nᵣ+nᵢₙ+1) MADDs and nᵣ nonlinear function evaluations and the exponential average computed with 2·(nᵢₙ+nᵣ) MADDs. For classification, nₒ·(nᵣ+nᵢₙ+1) MADDs and nₒ CMPs.

The weight matrices and biases need to be stored, in an embodiment, (nᵣ+nₒ)·(nᵣ+nᵢₙ+1) floats may be employed. Moreover, buffers for the hidden state, the running average and the classifier output, totaling nₒ+2·nᵣ+nᵢ floats may be employed.

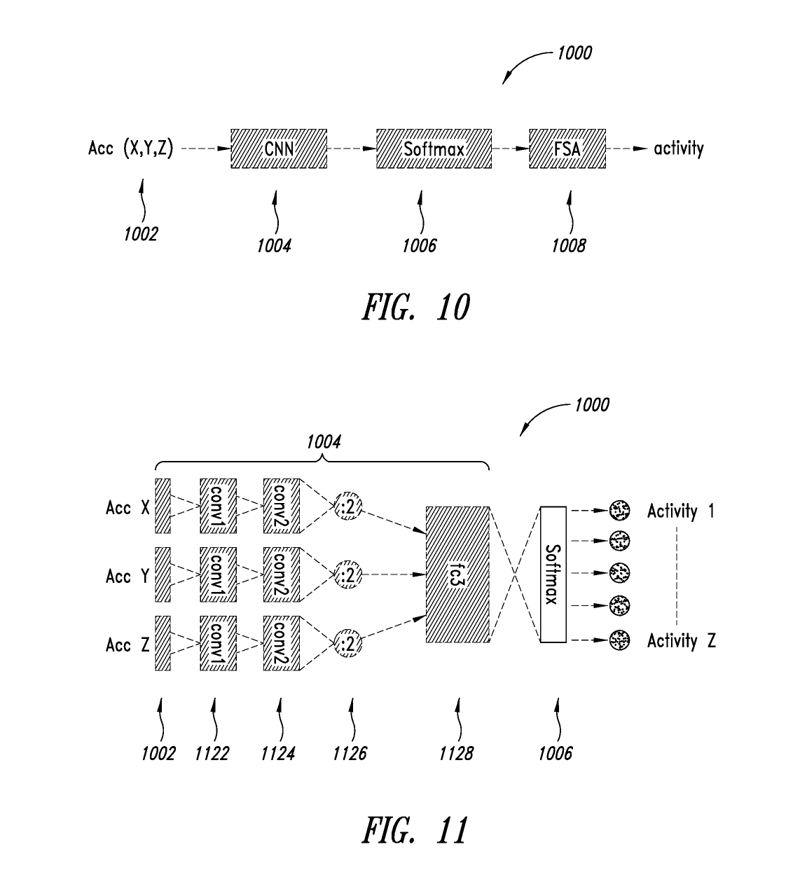

In the study, a convolutional neural network was employed. This model is feed-forward, that is, the output depends only on the input window and not on the temporal history of observations (in contrast with the ESN model). The CNN network in the study was built from m convolutional and n fully connected layers. The first m layers were applied to each acceleration axis separately; the resulting features were max pooled with size and stride of q and then fed to a Multi-Layer Perceptron (MLP) with p hidden neurons (fully connected to the MLP inputs) and r output neurons (one per class). The output of the CNN network was provided to a softmax layer (as a non-limiting example) giving the probability of each class and the class with the highest probability was selected as the prediction. To stabilize the prediction, a state filter (in this case, a finite state automata (FSA)) may be applied. FIG. 10 outlines an embodiment of a CNN network pipeline 1000, and FIG. 11 illustrates the embodiment 1000 of FIG. 10 in more detail. As illustrated in FIG. 10, Accelerometer data 1002 is provided to the CNN 1004, the output of the CNN is processed by a softmax layer 1006, and as illustrated, the prediction by the softmax layer 1006 is filtered by an optional FSA filter 1008.

FIG. 11 illustrates the CNN and Softmax portions of an embodiment of the CNN 1000 of FIG. 10. The convolutional layers 1122, 1124 learn filters of size k. The first layer 1122 generates m output features and the second layer 1124 generates again n features in output. The features are collected over a window of w samples and windows are separated by a step of s samples. The output of the convolutional layer 1124 is down-sampled by 2 (for example) in a pooling layer 1126. The MLP layer 1128 receives values in input and computes the hidden states. Then, the softmax layer 1006 computes the output as a softmax function with z output values, e.g., standing, walking, jogging, biking, driving, etc.

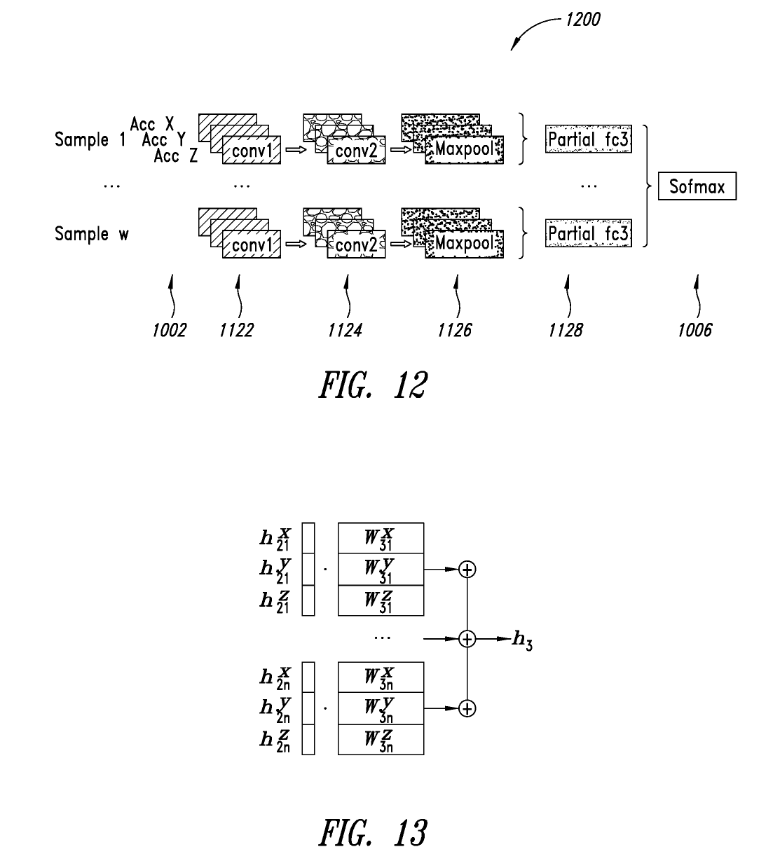

FIG. 12 illustrates an embodiment of a partial CNN computation method 1200 that may be employed by the embodiment 1000 of a CNN of FIG. 10. While the CNN model uses all of the samples from a window in order to make a decision, the samples can be processed in the convolutional layers as they arrive in order to interleave the computation with sensor reading (e.g., when implemented on an embedded platform) and reuse results between overlapping windows. Moreover, the output of the pooling 1126 and the fully-connected layer 3 (fc3) 1128 layers can be partially computed as soon as the filtered outputs of the convolutional layers are available for the current sample.

In the study, the three acceleration axes were processed separately through the two convolutional and the pooling layers, producing in output v values per sample. The values were multiplied by a slice W₃ᵢ of the weight matrix W₃ for the fc3 layer and accumulated on the fc3 output h₃ until all the samples 1, . . . , w of the current window were processed. The input values and the weights were re-ordered to match the order of the incoming values and to preserve the results of the matrix multiplication. FIG. 13 shows a logical diagram of the operations involved in the partial fc3 computation embodiment of the study.

In the study, the decision was computed by a softmax layer taking h₃ as input after all the samples in the window are processed. A single buffer may be employed as long as the step between windows is larger than 4·(k−1) where k is the size of the convolutional kernel; this condition ensures that the output of each window has no overlap. For shorter steps, more than one buffer and more than one fc3 output would be computed per sample, increasing memory and computational requirements.

FIG. 14 shows how the computational load is distributed, in the scenario where samples are read at a frequency of 16 Hz, and the partial computation of layers conv1 to fc3 (including an optional softmax) can be completed in less than 1/16^{th} of a second.

The CNN output can be affected by transient outliers, that is, predictions that for 1 or two windows give a different value from all the previous predictions because of, e.g., false positives. The outliers can be removed by examining the predictions in a window. This increases accuracy, at the cost of also increasing the latency in the prediction after an activity change. The outliers don't affect significantly the distribution of the predictions over a window, so the choice of an outlier removal mechanism may typically be a median filter with window size W.

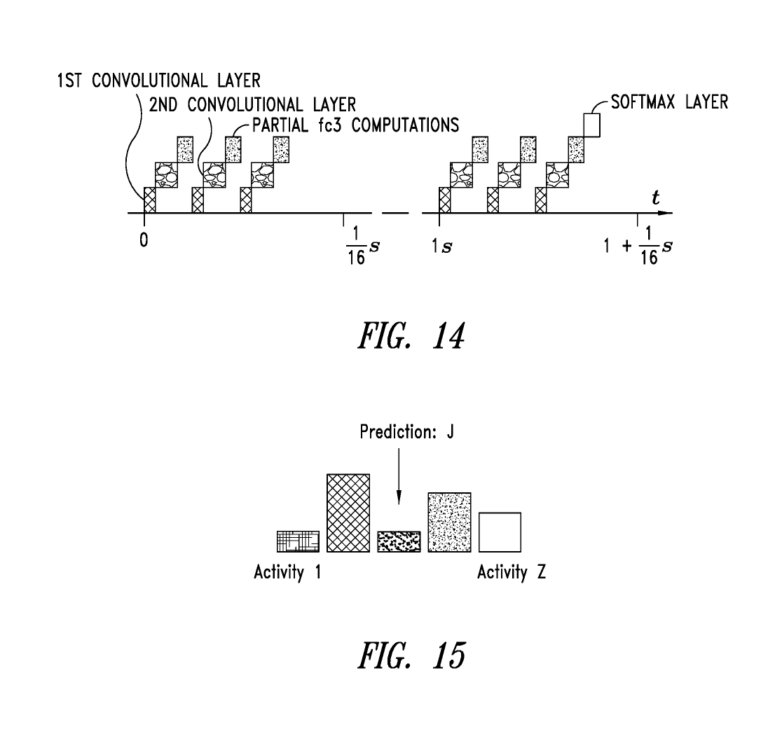

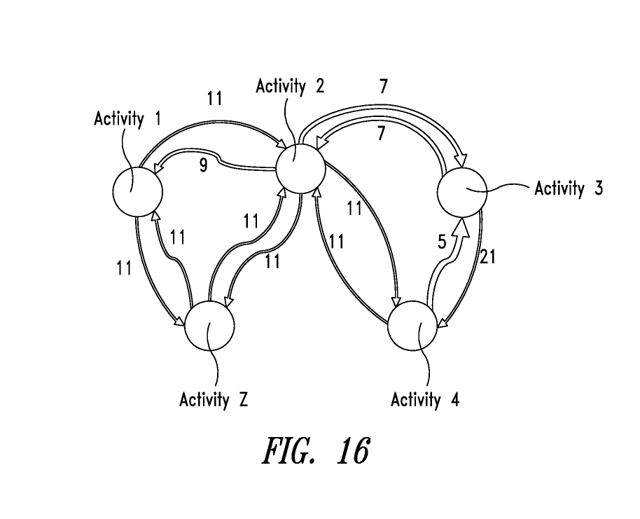

The use of a median filter, however, may present drawbacks. For example, all the state transitions are treated democratically by a median filter, even if some of the changes (e.g., jogging to driving) are implausible. In addition, an ordering in the activities given by the label codes is assumed, even if that order in meaningless, thus a prediction may be selected even if the prediction is underrepresented, because the prediction is in the middle of the distribution. FIG. 15 illustrates an example histogram of a label distribution on a window and a label selected by a median filter. In FIG. 15, the prediction is Activity 3, which is underrepresented in the window, but which is in the center of the window.

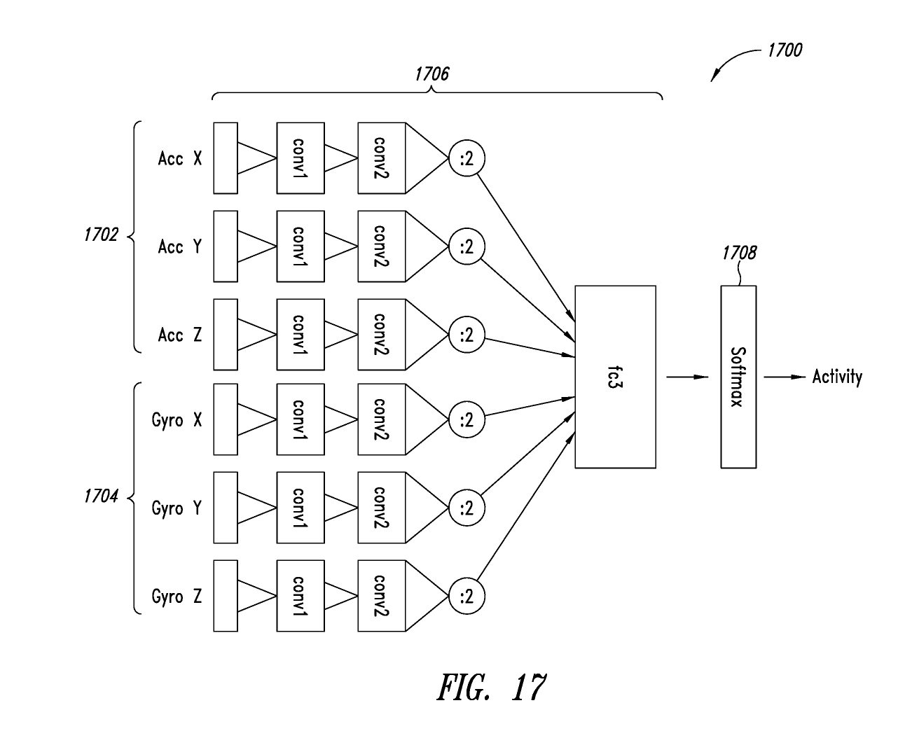

To facilitate addressing the drawbacks of a median filter, the likelihood of the transitions can be encoded in a variable window size, with longer windows selected for unlikely transitions. The transitions may be encoded in a Finite State Machine (FSM), also referred to as a Finite State Automata (FSA), where the states represent a confirmed prediction and the edges encode the window size to confirm a transition to another state. An example embodiment of an FSM (or FSA) is illustrated in FIG. 16. The dependence on the label order may be removed by using a majority filter, where a label is selected if at least 50% of the predictions in the window agree on a specific outcome.

The FSM (or FSA) may be implemented as a transition matrix T, where the element tᵢⱼ represents the window size from node i to node j and a special value (e.g., infinity) indicates that no transition is allowed. For transitions into the same state, a conventional value of 0 may be used. A queue of the last tₘₐₓ predictions may be maintained, where tₘₐₓ the maximum window size, and a transition from i to j is allowed if in the last tᵢⱼ predictions, at least

$$\frac{{t}_{ij}}{2} + 1 ( consensus threshold )$$

agree on the j label. If tᵢⱼ is the special value or no transition reaches the consensus threshold, the state is not changed.

It is noted that the neural network models may be trained end-to-end and do not require any specific parameter tuning, development of ad hoc rules, features, classification steps, etc. This may be advantageous in some embodiments. For example neural network embodiment may employ shorter design times for a classifier assuming a growing number of activities to be recognized from time to time. In addition, training tools optimized for powerful processor designs may significantly shorten the training times.

The Dataset 2.0 has a skewed class distribution, where the stationary class is overrepresented. As the stationary activity is much more likely than all the other activities, it makes sense to use a larger set of examples and adopt a training strategy that minimizes false negatives for the stationary class. For neural networks the same mechanism may be implemented explicitly (e.g. hard negative mining).

As mentioned above, the wearable device 110 of FIG. 1 may comprise one or more other sensors, such as one or more gyroscopes 160, in addition to one or more accelerometers 122. FIG. 17 illustrates CNN and Softmax portions of an embodiment of a CNN 1700, similar to the embodiment 1000 of FIGS. 10 and 11, but which processes accelerometer and gyroscope data. As illustrated in FIG. 17, Accelerometer data 1702 and gyroscope data 1704 are provided to the CNN 1706, the output of the CNN is processed by a softmax layer 1708. The prediction by the softmax layer 1708 may be filtered by an optional FSA filter (see FSA filter 1008 of FIG. 10). The training data set including acceleration and orientation data used to train the activity-classification model may be equalized with respect to the classifications. In some embodiments, additional activity-related data may be considered in the classification model, such as data from one or more physiological sensors, such as the physiological sensor 124 of FIG. 1, data from one or more position sensors, such as the position sensor 126 of FIG. 1, etc., and various combinations thereof.

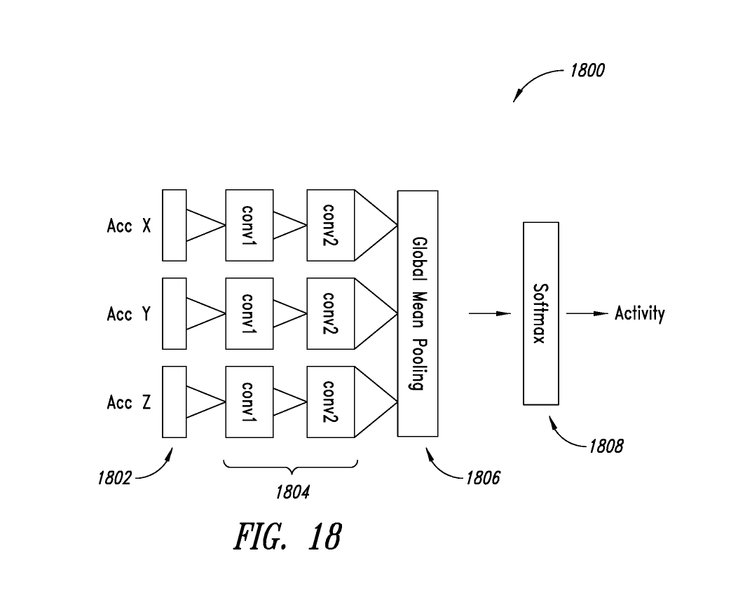

FIG. 18 illustrates CNN and Softmax portions of an embodiment of a CNN 1800, similar to the embodiment 1000 of FIGS. 10 and 11, where the fc3 layer has been replaced with a global mean pooling layer 1806. As illustrated in FIG. 18, Accelerometer data 1802 are provided to a series of convolutional layer 1804, an output of the series of convolutional layers 1804 is provided to the global mean pooling layer 1806. The global mean pooling layer selects the mean values across all the convolutional channels and the current window and provides these values to the softmax layer 1808. The prediction by the softmax layer 1808 may be filtered by an optional FSA filter (see FSA filter 1008 of FIG. 10). Some embodiments may employ other types of global pooling layers, such as a global maximum pooling layer, a global minimum pooling layer, etc. Using a global pooling layer instead of an fc3 layer may facilitate significantly reducing the number of parameters and operations. In some embodiments, using a global mean pooling layer instead of an fc3 layer may reduce the number of parameters by a factor of 10, and the number of operations by a factor of 2.

Some embodiments may take the form of or include computer program products. For example, according to one embodiment there is provided a computer readable medium including a computer program adapted to perform one or more of the methods or functions described above. The medium may be a physical storage medium such as for example a Read Only Memory (ROM) chip, or a disk such as a Digital Versatile Disk (DVD-ROM), Compact Disk (CD-ROM), a hard disk, a memory, a network, or a portable media article to be read by an appropriate drive or via an appropriate connection, including as encoded in one or more barcodes or other related codes stored on one or more such computer-readable mediums and being readable by an appropriate reader device.

Furthermore, in some embodiments, some of the systems and/or modules and/or circuits and/or blocks may be implemented or provided in other manners, such as at least partially in firmware and/or hardware, including, but not limited to, one or more application-specific integrated circuits (ASICs), digital signal processors, discrete circuitry, logic gates, standard integrated circuits, state machines, look-up tables, controllers (e.g., by executing appropriate instructions, and including microcontrollers and/or embedded controllers), field-programmable gate arrays (FPGAs), complex programmable logic devices (CPLDs), etc., as well as devices that employ RFID technology, and various combinations thereof.

The various embodiments described above can be combined to provide further embodiments. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method, comprising:
receiving activity-related data;
determining activity classifications based on the received activity-related data and an activity-classification model trained using a classification-equalized training data set; and
generating a classification signal based on the determined classifications.

2. The method of claim 1 wherein the classification-equalized training data set comprises a first class having a first sequence length and a number of samples N, and one or more additional classes each having a respective sequence length tⱼ and a respective number of samples Nⱼ which satisfy:
Nⱼ>N, for sequence length tⱼ; and
Nⱼ<N, for sequence length tⱼ−1.

3. The method of claim 2, comprising:
generating the classification-equalized training data set; and
training the activity-classification model.

4. The method of claim 1 wherein the determining activity classifications comprises extracting feature data based on the received accelerometer data.

5. The method of claim 1 wherein the determining comprises using an artificial neural network having a feed-forward architecture.

6. The method of claim 5 wherein the artificial neural network comprises a layered architecture including one or more of:
one or more convolutional layers;
one or more pooling layers; and
one or more softmax layers.

7. The method of claim 6 wherein the artificial neural network comprises a finite state machine.

8. The method of claim 1 wherein the determining comprises using an artificial neural network having a recurrent architecture.

9. The method of claim 8 wherein the artificial neural network comprises a finite state machine.

10. The method of claim 1 wherein the determining comprises using feature extraction and a random forest.

11. The method of claim 10 wherein the determining comprises applying and a temporal filter.

12. The method of claim 1 wherein the activity-related data comprises one or more of:
acceleration data;
orientation data;
geographical position data; and
physiological data.

13. A device, comprising:
an interface, which, in operation, receives one or more signals indicative of activity; and
signal processing circuitry, which, in operation:
determines activity classifications based on the received signals indicative of activity and an activity-classification model trained using a classification-equalized training data set; and
generates a classification signal based on the determined classifications.

14. The device of claim 13 wherein the classification-equalized training data set comprises a first class having a first sequence length and a number of samples N, and one or more additional classes each having a respective sequence length tⱼ and a respective number of samples Nⱼ which satisfy:
Nⱼ>N, for sequence length tⱼ; and
Nⱼ<N, for sequence length tⱼ−1.

15. The device of claim 14 wherein the signal processing circuitry comprises a feature extractor, and a temporal filter.

16. The device of claim 14 wherein the signal processing circuitry comprises an artificial neural network having a feed-forward architecture.

17. The device of claim 16 wherein the artificial neural network comprises a layered architecture including one or more of:
one or more convolutional layers;
one or more pooling layers; and
one or more softmax layers.

18. The device of claim 14 wherein the signal processing circuitry comprises an artificial neural network having a recurrent architecture.

19. The device of claim 14 wherein the signal processing circuitry, in operation:
generates the classification-equalized training data set; and
trains the activity-classification model.

20. The device of claim 13 wherein the signal processing circuitry comprises a finite state machine.

21. The device of claim 13 wherein the one or more signals indicative of activity comprise signals indicative of one or more of:
acceleration data;
orientation data;
geographical position data; and
physiological data.

22. A system, comprising:
one or more sensors, which, in operation, generates one or more activity-related signals; and
signal processing circuitry, which, in operation:
determines activity classifications based on activity-related signals and an activity-classification model trained using a classification-equalized training data set; and
generates a classification signal based on the determined classifications.

23. The system of claim 22 wherein the classification-equalized training data set comprises a first class having a first sequence length and a number of samples N, and one or more additional classes each having a respective sequence length tⱼ and a respective number of samples Nⱼ which satisfy:
Nⱼ>N, for sequence length tⱼ; and
Nⱼ<N, for sequence length tⱼ−1.

24. The system of claim 22 wherein the signal processing circuitry comprises a feature extractor and a temporal filter.

25. The system of claim 22 wherein the signal processing circuitry comprises an artificial neural network having a feed-forward architecture.

26. The system of claim 22 wherein the signal processing circuitry comprises an artificial neural network having a recurrent architecture.

27. The system of claim 22 wherein the one or more sensors include one or more of:
an accelerometer;
a gyroscope;
a position sensor; and
a physiological sensor.

28. A system, comprising:
means for providing activity-related data; and
means for generating an activity classification signal based on activity-related data and an activity-classification model trained using a classification-equalized training data set.

29. The system of claim 28 wherein the classification-equalized training data set comprises a first class having a first sequence length and a number of samples N, and one or more additional classes each having a respective sequence length tⱼ and a respective number of samples Nⱼ which satisfy:
Nⱼ>N, for sequence length tⱼ; and
Nⱼ<N, for sequence length tⱼ−1.

30. The system of claim 28 wherein the means for generating the activity classification signal comprises a memory and one or more processor cores, wherein the memory stores contents which in operation configure the one or more processor cores to generate the activity classification signal.
